(19) United States
(12) Patent Application Publication
(10) Pub. No.: US 2025/0171428 A1
(43) Pub. Date: May 29, 2025

(54) CRYSTALLINE FORMS OF 6-((5-METHYL-3-(6-METHYLPYRIDIN-3-YL)ISOXAZOL-4- YL)METHOXY)- N -(TETRAHYDRAPYRAN-4-YL)PYRIDAZINE-3-CARBOXAMIDE

(71) Applicant: Hoffmann-La Roche Inc., Little Falls, NJ (US)

(72) Inventors: André Marcel ALKER, Weil am Rhein (DE); Frank STOWASSER, Basel (CH)

(73) Assignee: Hoffmann-La Roche Inc., Little Falls, NJ (US)

(21) Appl. No.: 18/985,654

(22) Filed: Dec. 18, 2024

Related U.S. Application Data
(63) Continuation of application No. PCT/EP2023/066352, filed on Jun. 19, 2023.

(30) Foreign Application Priority Data
Jun. 20, 2022 (EP) 22179849.9

(51) Int. Cl. C07D 413/14

(52) U.S. Cl. CPC C07D 413/14

(57) ABSTRACT

Described herein are novel crystalline forms of 6-((5-methyl-3-(6-methylpyridin-3-yl) isoxazol-4-yl)methoxy)-N-(tetrahydropyran-4-yl)pyridazine-3-carboxamide, as well as pharmaceutical compositions comprising the same, processes for making them and their use in medical therapy.

### CROSS REFERENCE TO RELATED APPLICATIONS

This application is a continuation of International Patent Application No. PCT/EP2023/066352, filed Jun. 19, 2023, which claims priority to European Patent Application No. 22179849.9 filed Jun. 20, 2022, both of which are incorporated herein by reference in their entirety.

### FIELD OF THE INVENTION

This invention relates to novel crystalline forms of 6-((5-methyl-3-(6-methylpyridin-3-yl) isoxazol-4-yl)methoxy)-N-(tetrahydropyran-4-yl)pyridazine-3-carboxamide, to pharmaceutical compositions comprising said crystalline forms, to processes for making them and to their use in medical therapy.

### BACKGROUND OF THE INVENTION

It is important to identify forms of a drug that can be conveniently manufactured, formulated and administered to a patient.

Furthermore, in the manufacture of oral drug compositions, it is important that the drug is in a Form that provides reliable and reproducible plasma concentrations following administration to a patient.

Chemical stability, solid-state stability and “shelf life” of the drug substance are also particularly important factors. The drug substance, and compositions containing it, should ideally be capable of being effectively stored over appreciable periods of time, without exhibiting a significant change in the active component's physico-chemical characteristics (e.g. its chemical composition, density, hygroscopicity and solubility).

Moreover, it is also important to be able to provide a drug in a form that is as chemically pure as possible.

It is known that amorphous drug materials may present some problems in this regard. For example, such materials are typically difficult so handle and to formulate, provide for unreliable solubility, and are often found to be unstable and chemically impure.

The skilled person will therefore appreciate that, if a drug can be readily obtained in a stable crystalline form, many of the above problems may be solved. Thus, in the manufacture of commercially viable, and pharmaceutically acceptable, drug compositions, it is important, wherever possible, to provide the drug in a substantially crystalline and stable form. It is to be noted, however, that this goal is not always achievable. Indeed, based on molecular structure alone, it is not typically possible to predict what the crystallisation behaviour of a compound, either as such or in the form of a salt, will be. This can only be determined empirically.

WO2018104419, the entire Contents of which are incorporated herein by reference, discloses a Series of compounds that are positive allosteric modulators (PAM) of the GABA_{A} α5 receptor. WO2018104419 teaches that the compounds disclosed therein are potentially useful agents for use in the therapy of a number of medical conditions mediated by GABA_{A} α5 receptor activity, such as, Alzheimer's disease, mild cognitive impairment (MCI), age-related cognitive decline, negative and/or cognitive symptoms associated with schizophrenia, bipolar disorders, autism spectrum disorder (ASD), Angelman syndrome, Rett syndrome, Prader-Willi syndrome, epilepsy, post-traumatic stress disorder (PTSD), amyotrophic lateral sclerosis (ALS), and fragile-X disorder. One particular compound disclosed in WO2018104419 is 6-((5-methyl-3-(6-methylpyridin-3-yl) isoxazol-4-yl)methoxy)-N-(tetrahydropyran-4-yl)pyridazine-3-carboxamide (hereinafter Compound I), the structure of which is shown below.

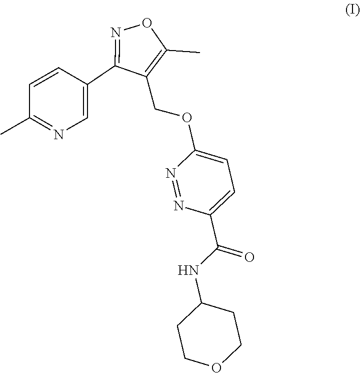 [SMILES: Cc1ccc(-c2noc(C)c2COc2ccc(C(=O)NC3CCOCC3)nn2)cn1]

Compound I is also known under the INN alogabat (WHO Drug Information, Vol. 35, No. 2, 2021, 366).

However, there is no disclosure in WO2018104419 of any crystalline forms of Compound I.

### SUMMARY OF THE INVENTION

In a first aspect, the present invention provides certain crystalline forms of 6-((5-methyl-3-(6-methylpyridin-3-yl) isoxazol-4-yl)methoxy)-N-(tetrahydropyran-4-yl)pyridazine-3-carboxamide (Compound I).

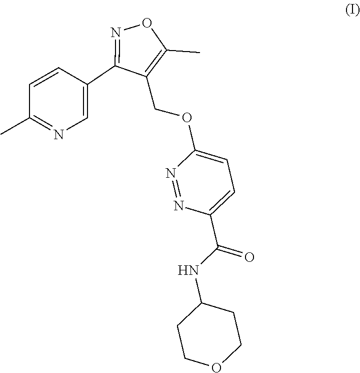 [SMILES: Cc1ccc(-c2noc(C)c2COc2ccc(C(=O)NC3CCOCC3)nn2)cn1]

In a further aspect, the present invention provides a pharmaceutical composition comprising any of the crystalline forms described herein.

In a further aspect, the present invention provides a crystalline form described herein for use as a medicament.

### BRIEF DESCRIPTION OF THE DRAWINGS

FIG. 1 shows a characteristic XRPD diffraction pattern of the Crystalline Form A of Compound 1.

FIG. 2 shows a characteristic XRPD diffraction pattern of the Crystalline Form B of Compound 1.

FIG. 3 shows a characteristic XRPD diffraction pattern of the Crystalline Form 4 of Compound 1.

### DETAILED DESCRIPTION OF THE INVENTION

### Definitions

The term “acceptable” with respect to a formulation, composition or ingredient, as used herein, means having no persistent detrimental effect on the general health of the subject being treated.

The terms “effective amount” or “therapeutically effective amount,” as used herein, refer to a sufficient amount of an agent being administered, which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a crystalline form as disclosed herein required to provide a clinically significant decrease in disease symptoms. The effective amount will be selected based on the particular patient and the disease level. It is understood that “an effect amount” or “a therapeutically effective amount” varies from subject to subject, due to variation in metabolism of drug, age, weight, general condition of the subject, the condition being treated, the severity of the condition being treated, and the judgment of the prescribing physician. In one embodiment, an appropriate “effective” amount in any individual case is determined using techniques, such as a dose escalation study. In some embodiments, the term “effective amount” or “therapeutically effective amount,” is used in reference to the crystalline forms described herein being administered, which will relieve to some extent one or more of the symptoms of the disease or condition being treated.

The term “prevention” as used herein refers to prophylactic treatments and includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

“Detectable amount” refers to an amount that is measurable using standard analytic methods (e.g. ion chromatography, mass spectrometry, NMR, HPLC, gas chromatography, elemental analysis, IR spectroscopy, inductively coupled plasma atomic emission spectrometry, USP<231> Method II, etc) (ICH guidances, Q2A Text on Validation of Analytical Procedures (March 1995) and Q2B Validation of Analytical Procedures: Methodology (November 1996)).

### Crystalline Forms

In one aspect, the present invention provides a crystalline Form A of 6-((5-methyl-3-(6-methylpyridin-3-yl) isoxazol-4-yl)methoxy)-N-(tetrahydropyran-4-yl)pyridazine-3-carboxamide (Compound I)

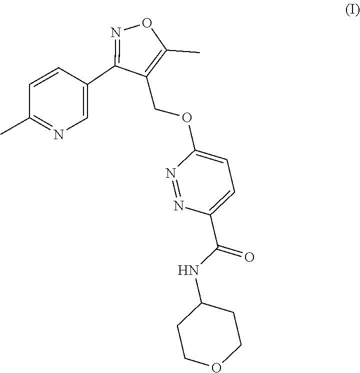 [SMILES: Cc1ccc(-c2noc(C)c2COc2ccc(C(=O)NC3CCOCC3)nn2)cn1]

which has an X-ray powder diffraction (XRPD) pattern comprising peaks at 18.47, 19.04, and 20.02 [° 2 Theta±0.2° 2 Theta, Cu Kα1 radiation (1.5406 Å)].

In one embodiment, said crystalline Form A has an X-ray powder diffraction (XRPD) pattern comprising peaks at 5.08, 16.20, 18.47, 19.04, and 20.02 [° 2 Theta±0.2° 2 Theta, Cu Kα1 radiation (1.5406 Å)].

In one embodiment, said crystalline Form A has an X-ray powder diffraction (XRPD) pattern comprising peaks at 5.08, 8.08, 14.87, 16.20, 18.47, 19.04, and 20.02 [° 2 Theta±0.2° 2 Theta, Cu Kα1 radiation (1.5406 Å)].

In one embodiment, said crystalline Form A has an X-ray powder diffraction (XRPD) pattern comprising peaks at 5.08, 8.08, 9.20, 10.17, 10.82, 12.75, 14.87, 15.37, 16.20, 17.42, 18.47, 18.80, 19.04, 19.66, 20.02, 21.10, 21.72, 22.39, 23.14, 23.80, 24.36, 24.57, 24.72, 25.03, 25.31, 25.57, 26.13, 26.48, 28.17, 28.37, 28.98, 29.12, 29.52, 30.14, and 31.46 [° 2 Theta±0.2° 2 Theta, Cu Kα1 radiation (1.5406 Å)].

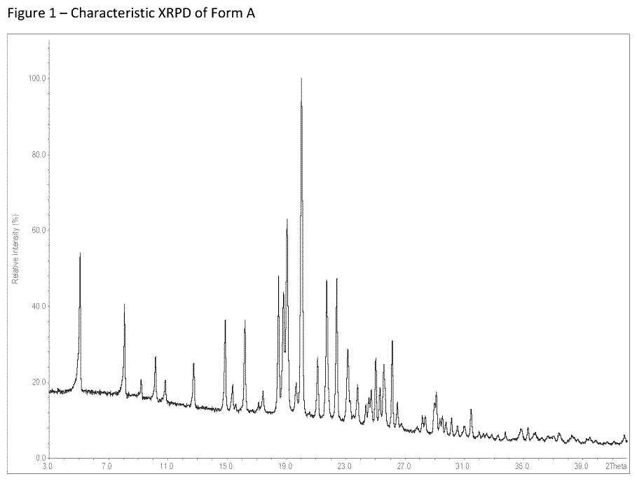

In one embodiment, said crystalline Form A has an X-ray powder diffraction (XRPD) pattern substantially the same as shown in FIG. 1.

In a further aspect, the present invention provides a crystalline Form B of 6-((5-methyl-3-(6-methylpyridin-3-yl) isoxazol-4-yl)methoxy)-N-(tetrahydropyran-4-yl)pyridazine-3-carboxamide (Compound I), which has an X-ray powder diffraction (XRPD) pattern comprising peaks at 9.33, 18.39, and 22.58 [° 2 Theta±0.2° 2 Theta, Cu Kα1 radiation (1.5406 Å)].

In one embodiment, said crystalline Form B has an X-ray powder diffraction (XRPD) pattern comprising peaks at 9.33, 17.42, 18.39, 19.97, and 22.58 [° 2 Theta±0.2° 2 Theta, Cu Kα1 radiation (1.5406 Å)].

In one embodiment, said crystalline Form B has an X-ray powder diffraction (XRPD) pattern comprising peaks at 9.33, 13.05, 15.73, 17.42, 18.39, 19.97, and 22.58 [° 2 Theta±0.2° 2 Theta, Cu Kα1 radiation (1.5406 Å)].

In one embodiment, said crystalline Form B has an X-ray powder diffraction (XRPD) pattern comprising peaks at 9.33, 11.87, 13.05, 15.73, 16.77, 16.92, 17.42, 18.39, 18.73, 19.62, 19.97, 21.08, 21.35, 22.46, 22.58, 23.75, 24.03, 24.92, 26.39, 26.79, 27.10, 27.94, 29.19, 29.61, 30.86, and 31.76 [° 2 Theta±0.2° 2 Theta, Cu Kα1 radiation (1.5406 Å)].

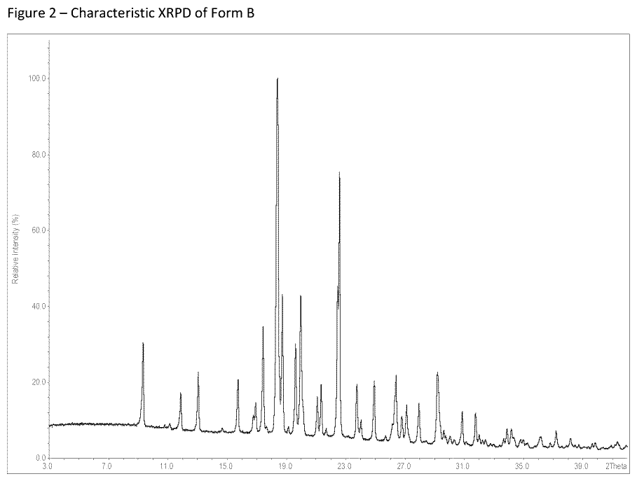

In one embodiment, said crystalline Form B has an X-ray powder diffraction (XRPD) pattern substantially the same as shown in FIG. 2.

In a further aspect, the present invention provides a crystalline Form 4 of 6-((5-methyl-3-(6-methylpyridin-3-yl) isoxazol-4-yl)methoxy)-N-(tetrahydropyran-4-yl)pyridazine-3-carboxamide (Compound I), which has an X-ray powder diffraction (XRPD) pattern comprising peaks at 6.64, 20.03, and 24.15 [° 2 Theta±0.2° 2 Theta, Cu Kα1 radiation (1.5406 Å)].

In one embodiment, said crystalline Form 4 has an X-ray powder diffraction (XRPD) pattern comprising peaks at 6.64, 16.76, 20.03, 24.15, and 27.68 [° 2 Theta±0.2° 2 Theta, Cu Kα1 radiation (1.5406 Å)].

In one embodiment, said crystalline Form 4 has an X-ray powder diffraction (XRPD) pattern comprising peaks at 6.64, 16.76, 20.03, 21.46, 22.20, 24.15, and 27.68 [° 2 Theta±0.2° 2 Theta, Cu Kα1 radiation (1.5406 Å)].

In one embodiment, said crystalline Form 4 has an X-ray powder diffraction (XRPD) pattern comprising peaks at 6.64, 7.66, 10.14, 13.30, 13.85, 15.37, 16.76, 17.26, 17.64, 17.95, 18.51, 18.91, 19.24, 20.03, 20.39, 21.46, 21.87, 22.20, 23.16, 23.47, 24.15, 25.37, 25.98, 26.57, 26.82, 27.08, 27.68, 29.04, 29.24, 29.81, 30.82, and 32.30 [° 2 Theta±0.2° 2 Theta, Cu Kα1 radiation (1.5406 Å)].

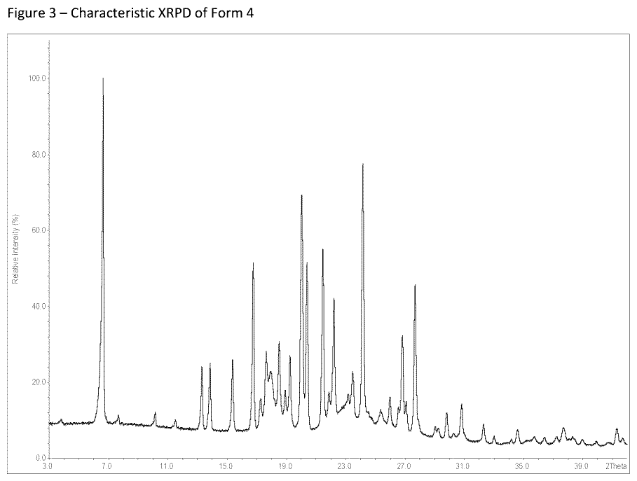

In one embodiment, said crystalline Form 4 has an X-ray powder diffraction (XRPD) pattern substantially the same as shown in FIG. 3.

### Preparation of Crystalline Forms

In one aspect, the present invention provides processes for preparing the crystalline forms described herein, wherein said processes are as outlined in the Examples. It is noted that solvents, temperatures and other reaction conditions presented in the Examples may vary.

In a further aspect, the present invention provides crystalline forms described herein, when obtained by the processes described in the Examples.

### Suitable Solvents

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene: carbon tetrachloride: 1,2-dichloroethane: 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetralin, toluene, 1,1,2-trichloroethene and xylene.

Class 3 solvents, which possess low toxicity, include: acetic acid, acetone, anisole, 1-butanol, 2-butanol, butyl acetate, tert-butylmethyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

In some embodiments, compositions comprising the crystalline forms described herein include a residual amount of an organic solvent(s). In some embodiments, compositions comprising the crystalline described herein include a detectable amount of an organic solvent(s). In some embodiments, compositions comprising the crystalline forms described herein include a residual amount of a Class 3 solvent. In some embodiments, the Class 3 solvent is selected from the group consisting of acetic acid, acetone, anisole, 1-butanol, 2-butanol, butyl acetate, tert-butylmethyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran. In some embodiments, the Class 3 solvent is selected from the group consisting of 1-butanol, 2-butanol, ethanol, 3-methyl-1-butanol, 2-methyl-1-propanol, 1-pentanol, 1-propanol, and 2-propanol. In some embodiments, the Class 3 solvent is ethanol or 1-propanol.

The crystalline forms described herein can exist in unsolvated as well as solvated forms with pharmaceutically acceptable solvents such as water, 1-propanol, ethanol, acetic acid and the like. For example, acetic acid solvates may be obtained by evaporative crystallization from a solvent mixture consisting of acetic acid and n-heptane.

### Amorphous Form

Despite the drawbacks that are generally associated with drug substances that are amorphous (vide supra), it may nevertheless be desirable to provide a drug substance in amorphous form, depending on its physico- and biochemical properties. Therefore, the present invention also provides amorphous 6-((5-methyl-3-(6-methylpyridin-3-yl) isoxazol-4-yl)methoxy)-N-(tetrahydropyran-4-yl)pyridazine-3-carboxamide (Compound I).

### Pharmaceutical Compositions/Formulations

Pharmaceutical compositions are formulated in a conventional manner using one or more physiologically acceptable carriers comprising excipients and auxiliaries which facilitate processing of the active compounds into preparations which are used pharmaceutically. Suitable techniques, carriers, and excipients include those found within, for example, Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975: Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

In one aspect, the present invention relates to a pharmaceutical composition comprising any of the crystalline forms described herein, and at least one pharmaceutically acceptable excipient.

In one embodiment, the pharmaceutical composition of the invention is for oral administration to a mammal.

In one embodiment, the pharmaceutical composition of the invention is a tablet.

In one embodiment, the pharmaceutical composition of the invention is a tablet comprising a kernel (core) and a coating.

In one embodiment, the tablet kernel comprises at least one pharmaceutically acceptable excipient selected from isomalt, microcrystalline cellulose, croscarmellose sodium, sucralose, colloidal silicon dioxide, and sodium stearyl fumarate.

In one embodiment said coating is Opadry II white 32F280008.

Contemplated pharmaceutical compositions provide a therapeutically effective amount of the crystalline forms described herein, enabling, for example, once-a-day, twice-a-day, three times a day, etc. administration. In one embodiment, pharmaceutical compositions provide an effective amount of the crystalline forms described herein, enabling once-a-day dosing.

In one embodiment, pharmaceutical compositions of the invention provide 1 mg to 50 mg, preferably 2 mg to 30 mg, more preferably 3 mg to 20 mg of the crystalline forms described herein. In a preferred embodiment, pharmaceutical compositions of the invention provide 3 mg, 4 mg, 10 mg or 20 mg of the crystalline forms described herein. In a particularly preferred embodiment, pharmaceutical compositions of the invention provide 3 mg of the crystalline forms described herein. In a particularly preferred embodiment, pharmaceutical compositions of the invention provide 4 mg of the crystalline forms described herein. In a particularly preferred embodiment, pharmaceutical compositions of the invention provide 10 mg of the crystalline forms described herein. In a particularly preferred embodiment, pharmaceutical compositions of the invention provide 20 mg of the crystalline forms described herein.

### Using the Crystalline Forms of the Invention

The compounds described herein possess valuable pharmacological properties for the treatment or prevention of medical conditions mediated by GABA_{A} α5 receptor activity.

In one aspect, the present invention provides the crystalline forms described herein for use as a medicament.

In one aspect, the present invention provides the crystalline forms described herein for use in the treatment or prevention of a medical condition mediated by GABA_{A} α5 receptor activity.

In one aspect, the present invention provides a method of treating or preventing a medical condition mediated by GABA_{A} α5 receptor activity in a mammal, said method comprising administering a therapeutically effective amount of a crystalline form described herein to said mammal.

In one aspect, the present invention provides the use of a crystalline form described herein in a method of treating or preventing a medical condition mediated by GABA_{A} α5 receptor activity in a mammal.

In one aspect, the present invention provides the use of a crystalline form described herein in the manufacture of a medicament for the treatment or prevention of a medical condition mediated by GABA_{A} α5 receptor activity in a mammal.

In one embodiment, said medical condition mediated by GABA_{A} α5 receptor activity is selected from Alzheimer's disease, mild cognitive impairment (MCI), age-related cognitive decline, negative and/or cognitive symptoms associated with schizophrenia, bipolar disorders, autism spectrum disorder (ASD), Angelman syndrome, Rett syndrome, Prader-Willi syndrome, epilepsy, post-traumatic stress disorder (PTSD), amyotrophic lateral sclerosis (ALS), and fragile-X disorder.

In one embodiment, said medical condition mediated by GABA_{A} α5 receptor activity is selected from Alzheimer's disease, mild cognitive impairment (MCI), age-related cognitive decline, negative and/or cognitive symptoms associated with schizophrenia, bipolar disorders, autism spectrum disorder (ASD), Angelman syndrome, Rett syndrome, Prader-Willi syndrome, epilepsy, post-traumatic stress disorder (PTSD), amyotrophic lateral sclerosis (ALS), and fragile-X disorder.

In a preferred embodiment, said medical condition mediated by GABA_{A} α5 receptor activity is selected from autism spectrum disorder (ASD) and Angelman syndrome.

In a particularly preferred embodiment, said medical condition mediated by GABA_{A} α5 receptor activity is autism spectrum disorder (ASD).

In a particularly preferred embodiment, said medical condition mediated by GABA_{A} α5 receptor activity is Angelman syndrome.

### EXAMPLES

The following examples are provided for illustration of the invention. They should not be considered as limiting the scope of the invention, but merely as being representative thereof.

### Example 1—Preparation of Crystalline Form A

Approx. 2.5 g of crude 6-((5-methyl-3-(6-methylpyridin-3-yl) isoxazol-4-yl)methoxy)-N-(tetrahydropyran-4-yl)pyridazine-3-carboxamide was dissolved in 50 mL of ethanol under reflux conditions. The resulting slightly brownish solution was allowed to cool down to ambient temperature, whereas seed crystals were added at 35 to 40° C. The beige suspension was stirred at ambient temperature for 1 h and at 0 to 5° C. for 1 h.

The crystals were isolated by filtration, rinsed with 10 mL of ethanol/n-heptane 1:1 and 10 ml of n-heptane. The solid was dried at 55° C./5 mbar yielding 1.9 g of voluminous white crystals.

### Example 2—Alternative Preparation of Crystalline Form A

306.9 mg of 6-((5-methyl-3-(6-methylpyridin-3-yl) isoxazol-4-yl)methoxy)-N-(tetrahydropyran-4-yl)pyridazine-3-carboxamide was dissolved in 6 mL of THF at ambient temperature. The vial was covered with a paper tissue to allow slow evaporation of the solvent at ambient temperature. After 1 day the solvent was evaporated to afford 6-((5-methyl-3-(6-methylpyridin-3-yl) isoxazol-4-yl)methoxy)-N-(tetrahydropyran-4-yl)pyridazine-3-carboxamide crystalline Form A.

### Example 3—Preparation of Crystalline Form B

330 g of 6-((5-methyl-3-(6-methylpyridin-3-yl) isoxazol-4-yl)methoxy)-N-(tetrahydropyran-4-yl)pyridazine-3-carboxamide was dissolved in 5 L of ethanol at 65° C. (turbid and light green solution with undissolved white particles). Filtration via carbon filter. Rinsed with 1 L of ethanol. The solvent was evaporated at 60° C. (bath temperature) and 200 mbar. The crystallization started when 1 L of ethanol was distilled off. The solvent was reduced until a thick suspension was left (additional 4.5 L of ethanol was distilled off). Under permanent stirring, the suspension was cooled down to ambient temperature. Afterwards stirring for 30 min at 0-5° C. The crystals were isolated by filtration, rinsed in portions with 0.6 L of cold ethanol (0-5° C.). The solid was dried at 60° C./2 mbar for 4 hours yielding 321 g white crystals.

Form B exhibited superior stability under all conditions tested (see Example 6).

### Example 4—Preparation of Crystalline Form 4

Approximately 300 mg of 6-((5-methyl-3-(6-methylpyridin-3-yl) isoxazol-4-yl)methoxy)-N-(tetrahydropyran-4-yl)pyridazine-3-carboxamide was dissolved in 6 mL of 1,4-dioxane at ambient temperature. The vial was covered with a paper tissue to allow slow evaporation of the solvent at ambient temperature. After 10 days, the solvent completely evaporated. The crystalline residue was identified as 6-((5-methyl-3-(6-methylpyridin-3-yl) isoxazol-4-yl)methoxy)-N-(tetrahydropyran-4-yl)pyridazine-3-carboxamide Form 4.

Form 4 was found to transform to Form B by providing a slurry of Form 4 in various solvents and solvent/water mixtures at temperatures ranging from 20° C. to 60° C.

At temperatures>110° C., Form 4 was found to transform to Form A.

### Example 5—XRPD

### Experimental Methodology

X-ray diffraction patterns were recorded at ambient conditions in transmission geometry with a STOE STADI P diffractometer (Cu Kα1 radiation, primary Ge-monochromator, Mythen IK silicon strip detector, angular range 3° to 42° 2 Theta, 0.02° 2 Theta step size, 20 seconds measurement time per step). The samples were prepared and analyzed without further processing (e.g. grinding or sieving) of the substance.

Measurement and evaluation of the X-ray diffraction data was done using WinXPOW software (STOE & Cie GmbH, Darmstadt, Germany).

### Results

The crystalline forms of Compound I were characterised by XRPD as described above. The unique XRPD peaks of the crystalline forms are presented in Table 1. Characteristic XRPD diffractograms of the crystalline forms are shown in FIGS. 1-3.

TABLE 1
XRPD peaks typical for Compound I

| Form A | | Form B | | Form 4 | |
|---|---|---|---|---|---|
| Peak position | Intensity | Peak position | Intensity | Peak position | Intensity |
| [° 2Theta] | [%] | [° 2Theta] | [%] | [° 2Theta] | [%] |
| 5.08 | 41 | 9.33 | 22 | 6.64 | 100 |
| 8.08 | 27 | 11.87 | 10 | 7.66 | 3 |
| 9.20 | 6 | 13.05 | 16 | 10.14 | 4 |
| 10.17 | 13 | 15.73 | 15 | 13.30 | 19 |
| 10.82 | 7 | 16.77 | 5 | 13.85 | 20 |
| 12.75 | 13 | 16.92 | 9 | 15.37 | 22 |
| 14.87 | 27 | 17.42 | 29 | 16.76 | 50 |
| 15.37 | 8 | 18.39 | 100 | 17.26 | 10 |
| 16.20 | 28 | 18.73 | 39 | 17.64 | 24 |
| 17.42 | 7 | 19.62 | 25 | 17.95 | 19 |
| 18.47 | 42 | 19.97 | 39 | 18.51 | 27 |
| 18.80 | 36 | 21.08 | 11 | 18.91 | 13 |
| 19.04 | 59 | 21.35 | 14 | 19.24 | 24 |
| 19.66 | 10 | 22.46 | 40 | 20.03 | 72 |
| 20.02 | 100 | 22.58 | 72 | 20.39 | 51 |
| 21.10 | 18 | 23.75 | 15 | 21.46 | 55 |
| 21.72 | 42 | 24.03 | 6 | 21.87 | 13 |
| 22.39 | 43 | 24.92 | 16 | 22.20 | 41 |
| 23.14 | 22 | 26.39 | 18 | 23.16 | 13 |
| 23.80 | 12 | 26.79 | 7 | 23.47 | 20 |
| 24.36 | 6 | 27.10 | 10 | 24.15 | 80 |
| 24.57 | 8 | 27.94 | 11 | 25.37 | 9 |
| 24.72 | 11 | 29.19 | 20 | 25.98 | 13 |
| 25.03 | 21 | 29.61 | 4 | 26.57 | 10 |
| 25.31 | 12 | 30.86 | 9 | 26.82 | 31 |
| 25.57 | 19 | 31.76 | 9 | 27.08 | 12 |
| 26.13 | 26 | | | 27.68 | 46 |
| 26.48 | 8 | | | 29.04 | 4 |
| 28.17 | 5 | | | 29.24 | 4 |
| 28.37 | 5 | | | 29.81 | 9 |
| 28.98 | 9 | | | 30.82 | 11 |
| 29.12 | 12 | | | 32.30 | 6 |
| 29.52 | 6 | | | | |
| 30.14 | 6 | | | | |
| 31.46 | 9 | | | | |

### Example 6—Competing Slurry Experiments

The competing long-term slurry equilibration experiments including Form A and Form B were performed in various solvents in the temperature range from 5° C. to 65° C.

All these experiments resulted in Form B, demonstrating its superior stability.

TABLE 2
Competing Slurry Experiments

| | Amount | | Volume | Equilibration | Equilibration | |
|---|---|---|---|---|---|---|
| | of API | Solvent | of solvent | temp. | time | XRPD Result |
| Starting material | [mg] | (mixture) | [mL] | [° C.] | [days] | wet |
| mixture of Form A | 51.7 | 1-propanol | 0.8 | 5 | 26 | Form B |
| and Form B | | | | | | |
| mixture of Form A | 49.6 | 1-propanol | 0.8 | 25 | 19 | Form B |
| and Form B | | | | | | |
| mixture of Form A | 49.6 | 1-propanol | 0.1 | 65 | 15 | Form B |
| and Form B | | | | | | |
| mixture of Form A | 49.7 | ethanol/water | 0.69 | 5 | 26 | Form B |
| and Form B | | (85/15; v/v) | | | | |
| mixture of Form A | 49.5 | ethanol/water | 0.35 | 25 | 19 | Form B |
| and Form B | | (85/15; v/v) | | | | |
| mixture of Form A | 50.4 | ethanol/water | 0.05 | 65 | 15 | Form B |
| and Form B | | (85/15; v/v) | | | | |
| mixture of Form A | 50.3 | dimethylformamide | 0.15 | 5 | 26 | Form B with |
| and Form B | | | | | | high orientation |
| | | | | | | effects |
| | | | | | | Form B + unknown |
| | | | | | | Mixture of |
| | | | | | | Form B with a |
| | | | | | | small ratio of |
| | | | | | | Form A |
| mixture of Form A | 55.8 | dimethylformamide | 0.085 | 25 | 19 | Form B |
| and Form B | | | | | | |
| mixture of Form A | 49.9 | dimethylformamide | 0.04 | 65 | 15 | Form B |
| and Form B | | | | | | |
| mixture of Form A | 49.8 | nitromethane | 0.62 | 5 | 26 | Form B |
| and Form B | | | | | | |
| mixture of Form A | 50.9 | nitromethane | 0.32 | 25 | 19 | Form B |
| and Form B | | | | | | |
| mixture of Form A | 50.6 | nitromethane | 0.05 | 65 | 15 | Form B |
| and Form B | | | | | | |
| mixture of Form A | 50.8 | methyl ethyl | 0.8 | 5 | 26 | From B |
| and Form B | | ketone | | | | |
| mixture of Form A | 49.4 | methyl ethyl | 0.67 | 25 | 19 | Form B |
| and Form B | | ketone | | | | |
| mixture of Form A | 49.9 | methyl ethyl | 0.1 | 65 | 15 | Form B |
| and Form B | | ketone | | | | |
| mixture of Form A | 50.3 | isopropyl | 0.8 | 5 | 26 | Form B |
| and Form B | | acetate | | | | |
| mixture of Form A | 50.3 | isopropyl | 0.8 | 25 | 19 | Form B |
| and Form B | | acetate | | | | |
| mixture of Form A | 50.5 | isopropyl | 0.4 | 65 | 15 | Form B |
| and Form B | | acetate | | | | |
| mixture of Form A | 50.3 | acetonitrile | 0.8 | 5 | 26 | Form B |
| and Form B | | | | | | |
| mixture of Form A | 50.3 | acetonitrile | 0.8 | 25 | 19 | Form B |
| and Form B | | | | | | |
| mixture of Form A | 50.1 | acetonitrile | 0.15 | 65 | 15 | Form B |
| and Form B | | | | | | |
| mixture of Form A | 50.0 | tetrahydrofuran | 0.3 | 5 | 26 | Form B |
| and Form B | | | | | | |
| mixture of Form A | 49.5 | tetrahydrofuran | 0.15 | 25 | 19 | Form B |
| and Form B | | | | | | |
| mixture of Form A | 50.8 | tetrahydrofuran | ~50 | 65 | 15 | Form B |
| and Form B | | | | | | |
| mixture of Form A | 49.3 | toluene | 0.8 | 5 | 26 | Form B |
| and Form B | | | | | | |
| mixture of Form A | 50.3 | toluene | 0.8 | 25 | 19 | Form B |
| and Form B | | | | | | |
| mixture of Form A | 50.2 | toluene | 0.25 | 65 | 15 | Form B |
| and Form B | | | | | | |
| mixture of Form A | 50.3 | chloroform | 0.05 | 5 | 26 | Form B |
| and Form B | | | | | | |
| mixture of Form A | 50.0 | chloroform | 0.02 | 65 | 15 | Form B |
| and Form B | | | | | | |
| mixture of Form A | 50.4 | chloroform | 0.04 | 25 | 14 | Form B |
| and Form B | | | | | | |
| mixture of Form A | 154.7 | 1-propanol | 0.4 | 65 | 14 | Form B |
| and Form B | | | | | 36 | Form B |
| mixture of Form A | 254.6 | methyl ethyl | 0.4 | 65 | 14 | Form B |
| and Form B | | ketone | | | 36 | Form B |
| mixture of Form A | 250.8 | tetrahydrofuran | 0.2 | 65 | 14 | Form B |
| and Form B | | | | | 36 | Form B |
| mixture of Form A | 125.8 | chloroform | 0.5 | 65 | 14 | Form B |
| and Form B | | | | | 36 | Form B |

1. Crystalline Form A of 6-((5-methyl-3-(6-methylpyridin-3-yl) isoxazol-4-yl)methoxy)-N-(tetrahydropyran-4-yl)pyridazine-3-carboxamide (Compound I) 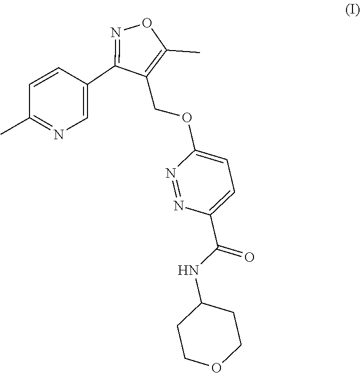 [SMILES: Cc1ccc(-c2noc(C)c2COc2ccc(C(=O)NC3CCOCC3)nn2)cn1]
which has an X-ray powder diffraction (XRPD) pattern comprising peaks at 18.47, 19.04, and 20.02 [° 2 Theta±0.2° 2 Theta, Cu Kα1 radiation (1.5406 Å)].

2. The crystalline Form A according to claim 1, which has an X-ray powder diffraction (XRPD) pattern comprising peaks at 5.08, 8.08, 9.20, 10.17, 10.82, 12.75, 14.87, 15.37, 16.20, 17.42, 18.47, 18.80, 19.04, 19.66, 20.02, 21.10, 21.72, 22.39, 23.14, 23.80, 24.36, 24.57, 24.72, 25.03, 25.31, 25.57, 26.13, 26.48, 28.17, 28.37, 28.98, 29.12, 29.52, 30.14, and 31.46 [° 2 Theta±0.2° 2 Theta, Cu Kα1 radiation (1.5406 Å)].

3. The crystalline Form A according to claim 1, which has an X-ray powder diffraction (XRPD) pattern substantially the same as shown in FIG. 1.

4. Crystalline Form B of 6-((5-methyl-3-(6-methylpyridin-3-yl) isoxazol-4-yl)methoxy)-N-(tetrahydropyran-4-yl)pyridazine-3-carboxamide (Compound I) 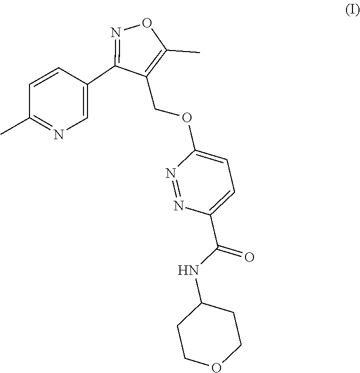 [SMILES: Cc1ccc(-c2noc(C)c2COc2ccc(C(=O)NC3CCOCC3)nn2)cn1]
which has an X-ray powder diffraction (XRPD) pattern comprising peaks at 9.33, 18.39, and 22.58 [° 2 Theta±0.2° 2 Theta, Cu Kα1 radiation (1.5406 Å)].

5. The crystalline Form B according to claim 4, which has an X-ray powder diffraction (XRPD) pattern comprising peaks at 9.33, 11.87, 13.05, 15.73, 16.77, 16.92, 17.42, 18.39, 18.73, 19.62, 19.97, 21.08, 21.35, 22.46, 22.58, 23.75, 24.03, 24.92, 26.39, 26.79, 27.10, 27.94, 29.19, 29.61, 30.86, and 31.76 [° 2 Theta±0.2° 2 Theta, Cu Kα1 radiation (1.5406 Å)].

6. The crystalline Form B according to claim 4, which has an X-ray powder diffraction (XRPD) pattern substantially the same as shown in FIG. 2.

7. Crystalline Form 4 of 6-((5-methyl-3-(6-methylpyridin-3-yl) isoxazol-4-yl)methoxy)-N-(tetrahydropyran-4-yl)pyridazine-3-carboxamide (Compound I) 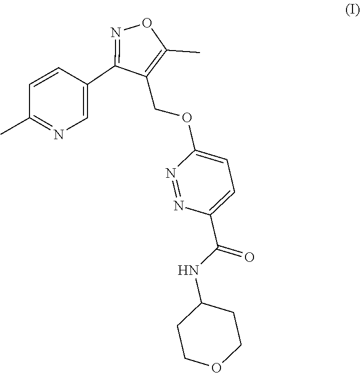 [SMILES: Cc1ccc(-c2noc(C)c2COc2ccc(C(=O)NC3CCOCC3)nn2)cn1]
which has an X-ray powder diffraction (XRPD) pattern comprising peaks at 6.64, 20.03, and 24.15 [° 2 Theta±0.2° 2 Theta, Cu Kα1 radiation (1.5406 Å)].

8. The crystalline Form 4 according to claim 7, which has an X-ray powder diffraction (XRPD) pattern comprising peaks at 6.64, 7.66, 10.14, 13.30, 13.85, 15.37, 16.76, 17.26, 17.64, 17.95, 18.51, 18.91, 19.24, 20.03, 20.39, 21.46, 21.87, 22.20, 23.16, 23.47, 24.15, 25.37, 25.98, 26.57, 26.82, 27.08, 27.68, 29.04, 29.24, 29.81, 30.82, and 32.30 [° 2 Theta±0.2° 2 Theta, Cu Kα1 radiation (1.5406 Å)].

9. The crystalline Form 4 according to claim 7, which has an X-ray powder diffraction (XRPD) pattern substantially the same as shown in FIG. 3.

10. A pharmaceutical composition comprising a crystalline form according to claim 1, and at least one pharmaceutically acceptable excipients, preferably wherein the pharmaceutical composition is in a form suitable for oral administration to a mammal.

11. A method of treating or preventing Alzheimer's disease, mild cognitive impairment, age-related cognitive decline, negative and/or cognitive symptoms associated with schizophrenia, bipolar disorders, autism spectrum disorder, Angelman syndrome, Rett syndrome, Prader-Willi syndrome, epilepsy, post-traumatic stress disorder, amyotrophic lateral sclerosis, and/or fragile-X disorder in a mammal, said method comprising administering a therapeutically effective amount of a crystalline form according to claim 1 to said mammal.
